(19) United States
(12) Patent Application Publication
(10) Pub. No.: US 2008/0097316 A1
(43) Pub. Date: Apr. 24, 2008

(54) Ultrasound catheter

(76) Inventors: Leonid Malinin, Newton, MA (US); Alexander Vladimirovich Kudryavtsev, Moscow (RU); Victor Efimovich Minaker, Moscow (RU); Pavel Yurievich Koblents, Saint Petersburg (RU); Paul Dicarlo, Middleboro, MA (US); Chris Elliott, Hopkinton, MA (US); Barbara Bell, Sudbury, MA (US)

Correspondence Address: FAY KAPLUN & MARCIN, LLP, 15O BROADWAY, SUITE 702, NEW YORK, NY, 10038, US

(21) Appl. No.: 11/507,180

(22) Filed: Aug. 21, 2006

(51) Int. Cl. A61M 37/00; A61M 25/00; A61M 5/31; A61B 18/18

(52) U.S. Cl. 604/131; 604/523; 604/247; 606/34

(57) ABSTRACT

A fluid infusion system comprises a flexible elongated body having a distal end, a proximal end and a fluid transport lumen extending therethrough and a fluid source having a connector for coupling to the proximal end to provide fluid to the fluid transport lumen in combination with an ultrasound energy source delivering ultrasound energy to the fluid to reduce a viscosity of the fluid and a waveguide directing the ultrasound energy to a desired region of the fluid.

### BACKGROUND OF THE INVENTION

Catheters are routinely used to form a semi-permanent path into the body to transfer fluids without repeatedly inserting a needle through the skin. The catheters may be used to infuse therapeutic compounds into the body and also to remove fluids therefrom. For example, a catheter may be used to drain fluids generated by infection, trauma, abscess or through normal metabolic function (e.g., urine).

Fluids infused through a catheter are often supplied from a pressurized source, such as a syringe, which forces the fluid into the catheter via a fluid connector. The speed of infusion is important, a faster infusion reduces the time required to administer a treatment and the cost of the procedure.

### SUMMARY OF THE INVENTION

In one aspect, the present invention is directed to a fluid infusion system comprising a flexible elongated body having a distal end, a proximal end and a fluid transport lumen extending therethrough and a fluid source having a connector for coupling to the proximal end to provide fluid to the fluid transport lumen in combination with an ultrasound energy source delivering ultrasound energy to the fluid to reduce a viscosity of the fluid and a waveguide directing the ultrasound energy to a desired region of the fluid.

### BRIEF DESCRIPTION OF THE DRAWINGS

FIG. 1 is a schematic side elevation view showing an injection syringe connected to a catheter;

FIG. 2 is a schematic side elevation view showing a first embodiment of an injection syringe connected to a catheter having an ultrasound waveguide according to the invention;

FIG. 3 is a schematic side elevation view showing a second embodiment of an injection syringe connected to a catheter with an ultrasound waveguide according to the invention;

FIG. 4 is a diagram showing a first embodiment of an ultrasound generator according to the invention;

FIG. 5 is a diagram showing a second embodiment of an ultrasound generator according to the invention;

FIG. 6 is a diagram showing an exemplary battery power source according to an embodiment of the invention;

FIG. 7 is a perspective view showing a catheter hub with a battery power source according to the invention;

FIG. 8 is a diagram showing flow rate as a function of the position of the ultrasound head for a syringe body, with the fluid under a gravity feed according to the invention; and

FIG. 9 is a diagram showing a catheter with two sources of ultrasound energy, according to another embodiment of the invention.

### DETAILED DESCRIPTION

The present invention may be further understood with reference to the following description and the appended drawings, wherein like elements are referred to with the same reference numerals. The invention relates to methods and devices for increasing the flow rate of fluids infused through a catheter. More specifically, the invention relates to the use of ultrasound energy to reduce the viscosity of a fluid being infused.

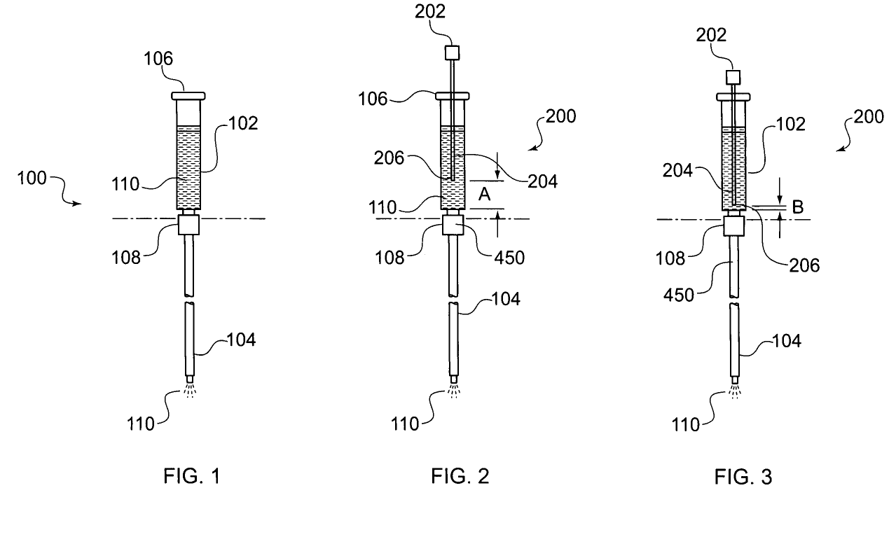

FIG. 1 shows an exemplary infusion apparatus 100 according to the present invention comprising a syringe 102 fluidly connected to a catheter 104 by a connector 108. Fluid 110 contained in the syringe 102 is pressurized by a piston 106 injected into the proximal end of the catheter 104 and ejected from the distal end thereof.

The embodiments of the present invention provide a method and apparatus increasing the flow rate of a fluid infused through a catheter by reducing the viscosity of the fluid, thus reducing the resistance to the flow through the syringe and the catheter. In one exemplary embodiment described in greater detail below, ultrasonic energy is applied to reduce the viscosity of the fluid which reduces friction with the surfaces of the catheter and other surfaces in contact with the fluid. The appropriate application of ultrasound also promotes maintaining a laminar flow through the catheter, which further reduces friction. Ultrasound energy may be applied to the fluid at the source, or at any other location in the hub or in the catheter. According to the invention, a source of ultrasound energy is incorporated into the catheter, for example, in an extension tube upstream of a hub used to connect the catheter to a fluid source.

FIG. 2 shows an infusion apparatus 200 according to an exemplary embodiment of the invention comprising a syringe 102 connected to a catheter 104 via a hub or fluid connector 108, for infusion of fluids 110. An ultrasound energy source 202 (e.g., an ultrasound crystal or array of crystals) near or in the syringe 102, provides high frequency, high energy ultrasound energy to the fluids 110 to reduce the viscosity thereof. The high frequency may be in the range of 25% above or below −22.65 kHz, while the high ultrasound energy may be in the range of 25% above or below 10 W. A waveguide 204 extends from the ultrasound energy source 202 into the body of the syringe 102, to convey the ultrasound energy into the fluids 110.

The distance that the waveguide 204 extends into the fluid 110 may be varied to obtain a desired viscosity reduction, controlling the increase in fluid flow rate. As shown in FIG. 2, the exemplary waveguide 204 is extended partially into the syringe 102 such that a distal end 206 thereof is separated by a distance “A” from a downstream end of the syringe 102 which is coupled to a fluid connector 108 while, in FIG. 3, the waveguide 204 is extended a greater distance into the syringe 102, so that the distal end 206 of the waveguide 204 is in close proximity to the fluid connector 108. In FIG. 3, the distal end 206 of the waveguide 204 is separated from the distal end of the syringe 102 by a distance “B” which is less than the distance “A”. The greater depth of the waveguide 204 in the fluids 110 allows the transfer of ultrasonic energy to a larger proportion of the fluids 110 increasing the reduction in viscosity. Thus, the distance between the bottom of the waveguide and the fluid connector 108 may be varied by a user to select a desired viscosity reduction for a specific application. A conventional mechanism may be used to advance and withdraw the source and/or waveguide into the fluids 110. Thus, a device according to this embodiment may be used in a range of applications with differing desired flow rates or fluid viscosities without replacing the syringe and/or the ultrasound source and waveguide combination.

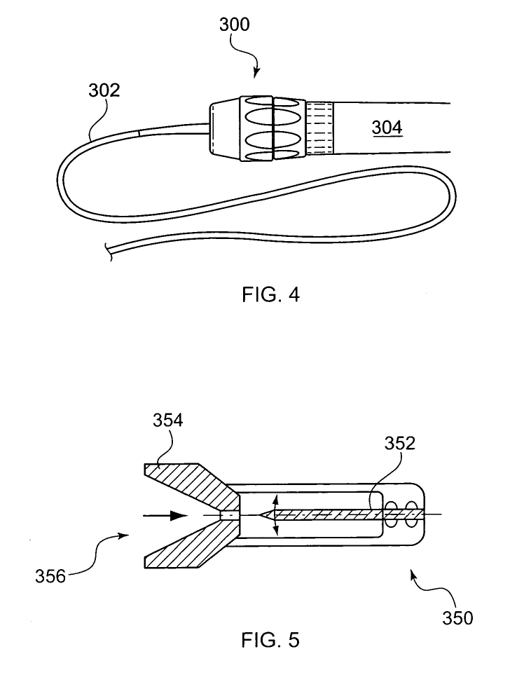

As would be understood by those skilled in the art, the source of ultrasound energy may be controlled to generate ultrasound energy having desired characteristics. For example, as shown in FIG. 4, a source 300 of ultrasound energy is an electro-mechanical component comprising a body 304 containing a vibration generating mechanism and an electric lead 302 through which power is supplied to the vibration generating mechanism. The electromechanical ultrasound generator 300 may, for example, be an ACUSON AcuNav™ 10F catheter, operating on a frequency of about 5 MHz, at a power of up to 25 W. In another example, the source 300 may be a Catheter for Ultrasound Trombolysis operating at a frequency of about 20 KHz at a power level of between about 16 W and 20 W.

In a different exemplary embodiment as shown in FIG. 5, the source of ultrasound energy may be a mechanical source 350 comprising a nozzle 354 that directs a flow of fluid 356 through the body of the device, which is shaped so that a plate 352 vibrates to generate the sound energy. The plate 352 may be made of metal, plastic, ceramic, or other similar material. As would be understood by those skilled in the art, ultrasonic energy generated by the mechanical source 350 is then transmitted to fluids 110 using, for example, using a waveguide as described above. The mechanical source 350 may be a Vortex whistle, operating in a frequency range of about 30 KHz to about 40 KHz, with a sound intensity of about 10 W/cm². Mechanical ultrasound sources have the advantage of being simple, inexpensive and easy to operate, while retaining an efficiency of up to 50%.

The ultrasound sources according to exemplary embodiments of the invention, utilize about 20 W. The time of operation necessary to inject a given amount of fluid e.g., 150 ml) may be derived by dividing the total volume by the flow rate. In the exemplary embodiment this comes to approximately 5 ml/sec with a total injection time of about 30 sec. Thus a battery powering a device for providing this level of flow must provide approximately 20 W for 30 seconds. This minimum power converts to 3.5V*5.7 A for 30 seconds. One suitable battery is the Fullymax Li—Po battery (FP353048P) which supplies a minimum capacity of 450 mAh at a voltage of 3.7 V, and a maximum discharge current of 6.8 A or up to 21 W.

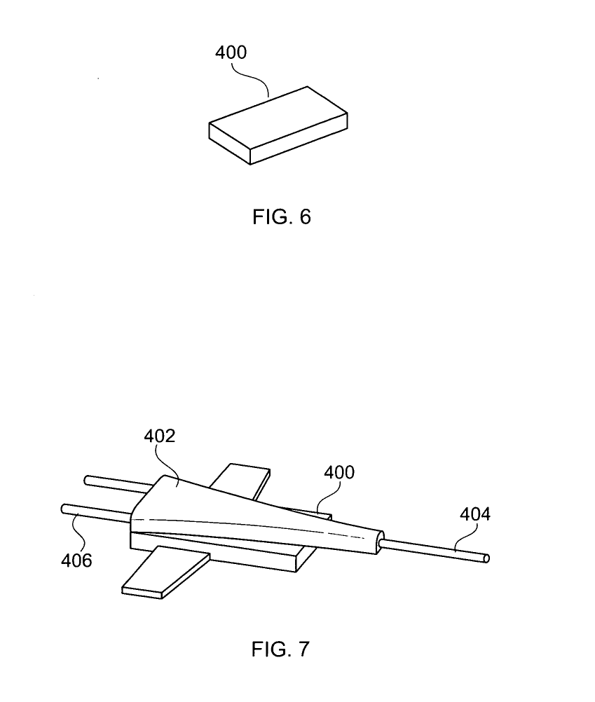

FIG. 6 shows an alternate battery 400 that may be used to power devices according to the invention. For example, as shown in FIG. 7, the battery 400 may be integrated into a hub 402 providing a fluid connection between a catheter 404 and a source of fluid through the conduits 406. As the battery 400 is incorporated in the fluid connector 402, there is no need to assemble an additional component while preparing the apparatus for infusion. In other exemplary embodiments, the battery 400 may be placed in another location on the hub 402, or in another component of the infusion device, in electrical contact with the source of ultrasound energy.

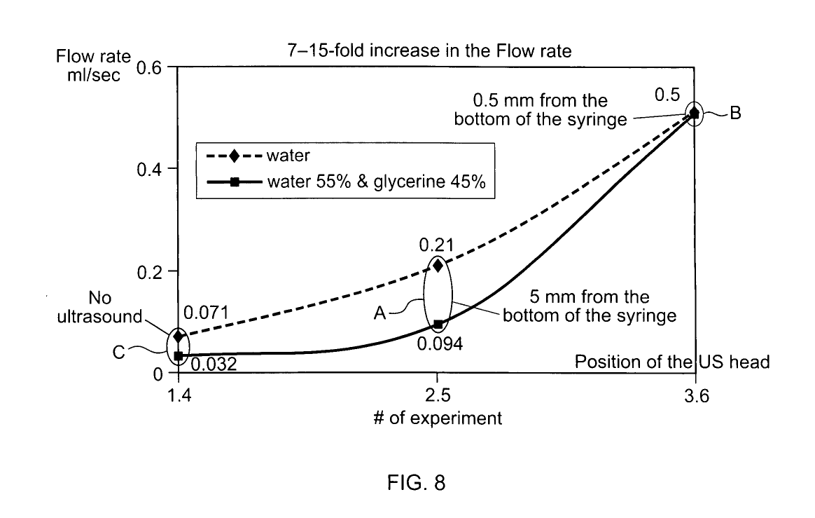

As indicated above, the waveguide 204 shown in FIGS. 2 and 3 may be inserted into the syringe 102 to a distance selected to obtain a desired reduction in viscosity. FIG. 8 shows a diagram of the relationship between the position of the distal end of the waveguide 204 (position axis) and the flow rate through the device, in ml/sec. The relationship is shown for water and for a fluid containing 55% water and 45% glycerin, to represent fluid properties typical of therapeutic infusion. Point A represents the conventional flow rate for the two fluids without the assistance of ultrasound energy.

Points A and B represent different locations of the ultrasound source/waveguide relative to the therapeutic fluid, with reference to the distal end of the syringe or the hub connecting the source to the catheter. Depending on the fluid used and the source location, an increase in flow rate of between about 7 and 15 times may be obtained using an exemplary ultrasound head installed in the hub of the catheter operating, for example, at a power of 10 W and a frequency of 22.65 kHz.

As shown in the diagram of FIG. 8, position A of the ultrasound head or waveguide, corresponding to a location of about 5.0 mm from the distal end of the syringe, gives flow rates for water with glycerin and for water only of 0.21 ml/sec and 0.094 ml/sec, respectively. These flow rates compare to unassisted flow rates of 0.071 mI/sec and 0.032 ml/sec, respectively. Position B of the ultrasound head or waveguide, corresponding to a location approximately 0.5 mm from the distal end of the syringe, yields a flow rate of 0.5 mI/sec for under a gravity feed with both fluids being considered. It is thus possible to select a location of the ultrasound head that produces a desired change in viscosity and, consequently, a desired flow rate.

According to the present invention, the use of ultrasound intensification to increase throughput of a therapeutic infusion may be applied to other areas of the catheter. For example, catheters often use safety valves to control the amount and direction of fluid therethrough. One type of valve, the pressure actuated safety valve (PASs Valve Technology) comprises a slitted membrane that allows fluid flow therethrough only when subjected to a fluid pressure greater than a predetermined threshold. However, even when the fluid pressure exceeds the threshold, the PASV restricts flow through the flow channel such that it is very beneficial to obtain a reduction in the viscosity of the fluid passing therethrough.

In one exemplary embodiment according to the invention, the PASV of a catheter comprises piezoelectric films or other ultrasound elements incorporated therein, or as a secondary membrane, to reduce the viscosity of fluid flowing therethrough increasing flow rate through the catheter. Piezoelectric films added to the PASV may also act as a pump to further increase flow rate through catheter. Furthermore, the viscosity reduction function and the pump function of the piezoelectric films may be used concurrently, to further increase flow through the catheter. Once the PASV is opened, the frequency could cause a peristaltic action.

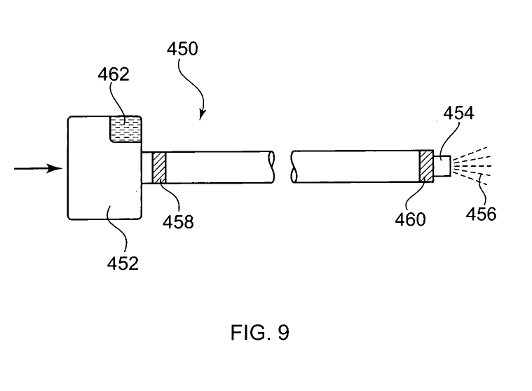

In a further exemplary embodiment of the present invention, transducers may be located at different locations along the longitudinal axis of the catheter. If desired, the transducers may be set to operate at slightly different frequencies. For example, two acoustic transducers may be used to generate a beat frequency that helps drive flow through the catheter. The exemplary catheter 450 shown in FIG. 9 comprises a first acoustic transducer 458 disposed near a proximal hub 452 and a second acoustic transducer 460 disposed more distally at a location 454. The beat frequency resulting from driving the transducers out of phase increases the flow rate of the fluid 456. The beat frequency will depend on the size of the catheter, but may range from 1 kHz to 100 kHz.

The transducers according to the present invention may be placed at wall locations prone to eddy currents (i.e. just distal to the suture wing). The transducer may thus break up the flow pattern and help to preserve a laminar flow, resulting in greater flow rate. For example, a transducer 462 may be disposed along the wall of the hub 452.

In an alternate embodiment of the invention, micro-electro mechanical systems (MEMS) may be embedded in the catheter, valve and/or fluid source to provide additional functionality to the device. For example, MEMS may be embedded into piezoelectric films such as films 458, 460 and 462 to serve as sources of ultrasound energy. The MEMS preferably sense and control the flow rate of the fluids 110 through the catheter 450 to maintain the flow rate at an optimum level which may be further increased with respect to the embodiments described above. For example, the MEMS may sense the condition(s) of the infusion and control operation of the ultrasound source(s) based on the sensed condition(s). If the sensed viscosity is too high, the ultrasound source(s) may be activated to reduce the viscosity to a desired level.

As would be understood by those skilled in the art, a high intensity focused ultrasound (HIFU) device may be used to increase the amplitude of the ultrasound energy delivered. For example, the source 202 may be a HIFU source generating energy which is more focused on a desired region of the fluid flow and which, consequently, affects the infusion flow rate more than is possible with non-focused ultrasound energy. For example, focused ultrasound energy may be directed to an especially turbulent flow region to reduce turbulence and minimize resistance to the passage of the fluid through the region.

The present invention has been described with reference to specific embodiments. However, other embodiments may be devised that are applicable to other types of catheters and procedures. Accordingly, various modifications and changes may be made to the embodiments, without departing from the broadest spirit and scope of the present invention as set forth in the claims that follow. The specification and drawings are accordingly to be regarded in an illustrative rather than restrictive illustrative rather than restrictive sense.

What is claimed is:

1. A fluid infusion system comprising:
a flexible elongated body having a distal end, a proximal end and a fluid transport lumen extending therethrough;
a fluid source having a connector for coupling to the proximal end to provide fluid to the fluid transport lumen;
an ultrasound energy source delivering ultrasound energy to the fluid to reduce a viscosity of the fluid; and
a waveguide directing the ultrasound energy to a desired region of the fluid.

2. The fluid infusion system according to claim 1, wherein the waveguide directs the ultrasound energy to the fluid source.

3. The fluid infusion system according to claim 1, wherein the fluid source is a hand operated syringe.

4. The fluid infusion system according to claim 3, wherein the waveguide extends into the syringe.

5. The fluid infusion system according to claim 4, wherein a position of a distal end of the waveguide within the syringe is variable by a user of the system.

6. The fluid transport system according to claim 3, wherein a distal end of the waveguide extends to between about 0.5 cm and 5.0 cm of a distal end of a fluid chamber of the syringe.

7. The fluid transport system according to claim 1, wherein the ultrasound energy source is disposed in a hub coupled to the flexible elongate body.

8. The fluid transport system according to claim 1, further comprising a PASV mounted within the fluid transport lumen, the ultrasound energy source being located adjacent the PASV.

9. The fluid transport system according to claim 1, wherein the ultrasound energy source comprises one of a mechanical ultrasound generator, an electro-mechanical ultrasound generator and a piezoelectric element.

10. The fluid transport system according to claim 1, wherein the ultrasound energy source comprises at least two ultrasound generators operating at different frequencies.

11. The fluid transport system according to claim 10, wherein the at least two ultrasound generators are disposed at substantially opposite ends of the fluid source.

12. The fluid transport system according to claim 1, wherein the ultrasound energy source operates at between about 5 KHz and about 40 KHz.

13. The fluid transport system according to claim 1, wherein the ultrasound energy source operates at a power of between about 15 W and about 25 W.

14. A method of infusing fluids to a body comprising:
introducing a fluid delivery lumen to a desired location within the body; and
delivering ultrasound energy to a fluid to be supplied to the lumen to reduce a viscosity of the fluid.

15. The method according to claim 14, further comprising coupling to a source of the ultrasound energy a waveguide operatively directing the ultrasound energy to a desired region of the fluid.

16. The method according to claim 14, wherein the fluid is introduced to the fluid delivery lumen via a hand operated syringe.

17. The method according to claim 15, wherein the waveguide directs the ultrasound energy to a source of the fluid before it enters the fluid delivery lumen.

18. The method according to claim 17, wherein the waveguide extends into the fluid source.

19. The method according to claim 14, further comprising providing ultrasound energy comprises two ultrasound generators operating at difference frequencies.

20. The method according to claim 19, further comprising a battery integral with a coupling between the fluid transport lumen and a source of the fluid.

21. The method according to claim 14, wherein a source of ultrasound energy operates at a frequency of between about 5 KHz and about 40 KHz.

22. The method according to claim 14, wherein a source of ultrasound energy operates at a power level of between about 15 W and 25 W.
